(19) United States
(12) Patent Application Publication
(10) Pub. No.: US 2022/0031902 A1
(43) Pub. Date: Feb. 3, 2022

(54) Dental/medical operatory ventilation system

(71) Applicant: Bradley Evan Cohn, Pepper Pike, OH (US)

(72) Inventor: Bradley Evan Cohn, Pepper Pike, OH (US)

(21) Appl. No.: 17/300,492

(22) Filed: Jul. 19, 2021

Related U.S. Application Data
(60) Provisional application No. 63/058,566, filed on Jul. 30, 2020.

(51) Int. Cl. A61L 9/12; A61L 9/01

(52) U.S. Cl. CPC A61L 9/122; A61G 15/10; A61L 9/01

(57) ABSTRACT

A ventilation system for removal of aerosols including bacteria and viruses such as the COVID-19 Coronavirus, and airborne debris from dental/medical operatories is provided. This ventilation system is comprised of (1) a stationary hood, secured at a specific distance above a patient that pulls contaminated air from the operatory, (2) ducting that carries the contaminated air away from the operatory, (3) a UV Ozonator that treats the air and cleans it of viral and bacterial aerosols and airborne debris before (4) expelling it to the outside through a specified length of terminal duct. The combination of certain embodiments in this invention create an effective means of aerosol and airborne debris removal in dental/medical operatories or other indoor spaces.

### BACKGROUND OF THE INVENTION

Restorative Dentistry involves prepping teeth with air and water spray. This procedure produces aerosols containing dust and debris that are removed with a high-volume evacuator, that is able to contain much of the aerosols, but not all. Because of the COVID-19 pandemic, there is a need to remove virtually all aerosols in the dental/medical operatory, including droplets that may contain the COVID-19 virus, for the safety of patients, doctors, and staff. The COVID-19 pandemic has brought renewed awareness of the need for improved operatory ventilation because of the possibility of aerosols spreading droplets containing the COVID-19 virus. The COVID-19 pandemic has been devastating to American families and small businesses. In fact, since the start of the pandemic, dental and other medical offices have accommodated many restrictions and safety precautions recommended by the CDC. However, even with all safety precautions in place, many people are fearful of visiting dental and medical offices, because of their fear of being exposed to the COVID-19 virus. There are widespread reports that people are staying home rather than taking care of dental and medical concerns. Currently, there are no ventilation systems available for dental/medical operatories. Many practices use Air Cleaners, HVAC systems, or portable units for filtration that generally distribute filtered air throughout indoor spaces and do not solve the problem of aerosol spread. The purpose of this invention is to produce a safe environment for patients, personnel, and dentists while practicing dentistry.

### SUMMARY OF THE INVENTION

With the herein disclosed invention, it is now possible to remove virtually all aerosolized bacteria, viruses, and debris from a dental/medical operatory or other space. This invention will improve safety in dental/medical operatories by removing aerosols from the air, treating the air to remove bacteria and viruses, including the COVID-19 virus, and exhausting cleaned air to the outside. In this design, the ceiling dental lamp is removed as well as the drop ceiling tiles to uncover the dental light support. The support structure for the overhead dental lamp is ideal for the support structure for the hood. By placing a 54″ to 60″ hood no more than 88 inches above the floor, a minimum 1200 cfm hood with attachments, effectively removes aerosols and debris from a dental/medical operatory. Since this invention makes use of existing support structures and existing electrical code configurations in the ceiling of a dental/medical operatory, it is a practical addition to any dental/medical facility, or other space requiring ventilation.

### BRIEF DESCRIPTION OF DRAWINGS

FIG. 1. Ventilation hood over dental chair that has lighting to replace track lighting for a dentist and adds ventilation over the work area. Placement does not impede any other operations in the dental operatory because of its position. Dental x-rays can be taken freely and equipment can be moved in and out. The position of the hood allows for the evacuation of airborne particles and aerosols during dental procedures.

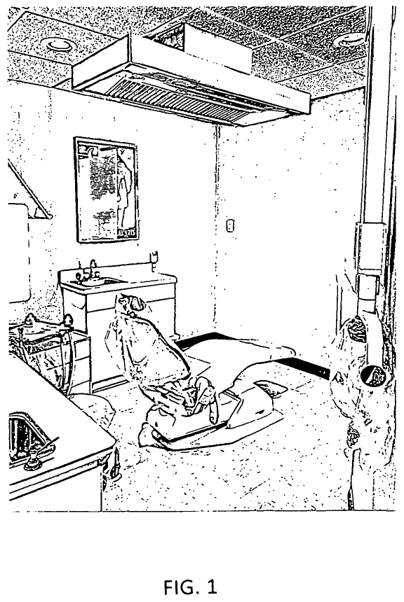

FIG. 2. Side view of hood in the dental operatory.

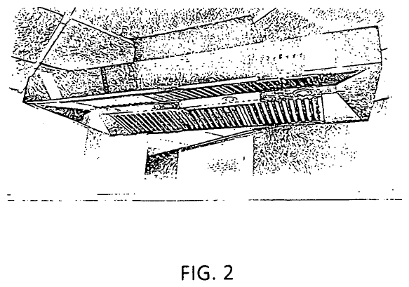

FIG. 3. Front view of hood in dental operatory.

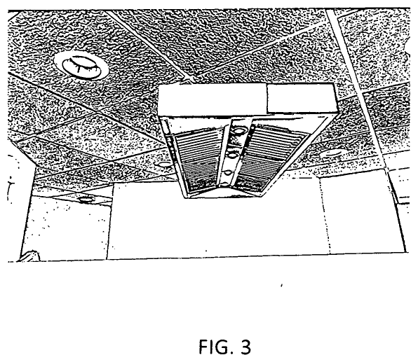

FIG. 4. Diagram of the ultra-violet 8″ In-Line DUCT Ozonator encountered by air before leaving the vent and being expelled to the outside of the building.

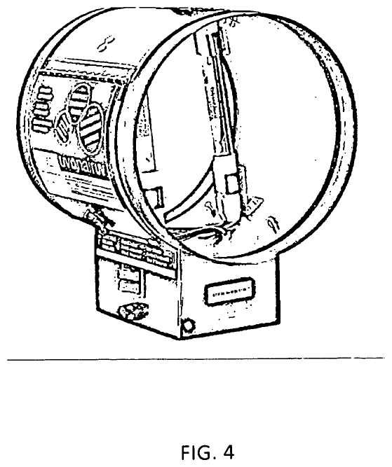

FIG. 5. Diagram of sequence of attachments for the Dental/Medical Operatory Ventilation System. A 15 ft. distance from the UV Ozonator to the outside is required for this unit.

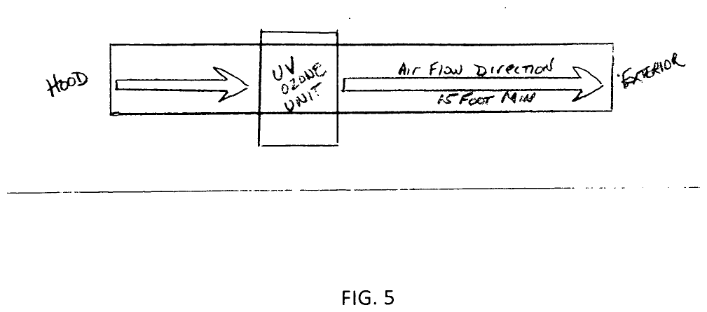

### DETAILED DESCRIPTION OF INVENTION

This invention consists of a hood attached to venting, and a UV Ozonator with a specified amount of terminal venting that opens to the outside of the building. Currently, this inventor has installed Zline Model #KECOMi-54 in. and 60 in. ventilation hoods that are 88″ from the floor and directly overhead the dental chair and patient. The positioning is important so as not to impede dental x-ray equipment or movement around the operatory. With placement at this specific height with these units, dental x-rays can be taken freely and equipment can be moved in and out of the operatory. The position of the hood allows for efficient and effective evacuation of aerosols and particles during dental procedures, while not interfering with movement in the operatory during procedures.

There is a fan in the hood that draws air from above the patient, wherein the air travels through the attachments of the Dental/Medical Operatory Ventilation System and provides a clean and uncontaminated environment for the patient, the dentist and dental assistants. In addition, other patients in the office, and even tenants in the building do not need to worry about contaminated air from the dental office. To operate the hood, there are 6 buttons located on the side of the hood. Button 1 is the on/off button; buttons 2-5 are the speeds of the exhaust fan; and button 6 is the on/off button for the light. The UV Ozonator is turned on separately, and this inventor placed the UV Ozonator on/off switch at a convenient location on the wall in the dental operatory.

The hood is powered by the same power supply as an overhead dental light, which makes it easy to install the hood since the electrical supply is already intact. Because the hood has the same electrical requirements as the overhead dental lamp, this invention can be easily installed in any dental office. Specifications for a dental light require that it must be on its own 20 amp circuit. Coincidentally, both the dental light and the ventilation hood have the same requirements. The hood contains a motor with a range of speeds from 280 cfm to the highest speed of 1200 cfm, that effectively removes aerosols from a dental/medical operatory.

The overhead dental lamp is removed for installation of the hood, as the hood contains lights that will replace the overhead dental lamp. The structure in the ceiling of the operatory that supported the dental lamp will also support the exhaust hood with minor modifications. The hood lights can be adjusted and directed by the dentist over the work area and thus replaces the need for the overhead dental lamp. Since this invention makes use of existing support structures with only a minor modification in the ceiling of a dental or medical operatory, it is a practical addition to any dental or medical facility.

This invention consists of an exhaust hood with certain specifications and provides exhaust of contaminated air from the dental or medical operatory. The hood and attachments replace the overhead lamp and is secured using a minor modification of the support structures in the ceiling as used for the overhead lamp. The air can travel through an 8″ to 12″ duct to a UV Ozonator, then the air travels through 15′ of the 8″ to 12″ ducting before being expelled to the outside. There is an on/off switch for the UV Ozonator on the wall of the dental/medical operatory.

The size of the UV Ozonator that is currently being used requires 15′ of 8″ duct before expelling the treated air to the outside. Different configurations of the number of operatories that require ventilation will dictate the size of ducting necessary. It has been determined that the minimum duct size is 8″. The UV Ozonator kills 99.99% of viruses, including the COVID-19 Coronavirus. The unit has two speeds, low and high. In this case, this inventor has the unit set to high, and so the on/off switch turns it on to the high setting, and off. According to the UV Ozonator company that produces this unit, it is also nontoxic to the environment, since it does not produce hazardous waste. As a bonus, the UV Ozonator also controls odors, and as advertised, this unit destroys odors rather than masking them. Therefore, treated air that is expelled to the outside will be free from bacteria, viruses and odors.

I claim:

1. An effective ventilation system for dental/medical operatories, or other indoor spaces comprised of:
a ventilation hood, a UV Ozonator, and 8″ to 12″ duct;
a 54″ or 60″ hood is placed no higher than 88 inches from the floor;
a hood should be at least 1200 cfm to effectively remove contaminated aerosols;
hood should be placed above the patient chair;
exhaust air travels through the 8″ to 12″ duct;
air passes through the UV Ozonator effectively destroying unwanted contaminants;
air continues through 15′ of 8″to 12″ duct and is treated and cleaned of contaminants;
treated air is expelled to the outside;
placement of the hood does not hinder movement about the room.
